(19) United States
(12) Patent Application Publication
(10) Pub. No.: US 2010/0297686 A1
(43) Pub. Date: Nov. 25, 2010

(54) DEVICES FOR INTRACELLULAR SURFACE-ENHANCED RAMAN SPECTROSCOPY

(75) Inventors: Yury Gogotsi, Warminister, PA (US); Gennady Friedman, Richboro, PA (US); Elina Vitol, Drexel Hill, PA (US)

Correspondence Address: WOODCOCK WASHBURN LLP, CIRA CENTRE, 12TH FLOOR, 2929 ARCH STREET, PHILADELPHIA, PA, 19104-2891, US

(73) Assignee: DREXEL UNIVERSITY, Philadelphia, PA (US)

(21) Appl. No.: 12/782,885

(22) Filed: May 19, 2010

Related U.S. Application Data
(60) Provisional application No. 61/180,160, filed on May 21, 2009.

(51) Int. Cl. C12Q 1/02; G01J 3/44; G01N 33/48

(52) U.S. Cl. 435/29; 356/301; 436/63

(57) ABSTRACT

Provided are surface-enhanced Raman spectroscopy (SERS) devices suitable for intra-subject (e.g., intracellular) observation, which devices may be of nanoscale size. Also provided are related SERS analysis methods.

### RELATED APPLICATIONS

The present application claims the benefit of U.S. application Ser. No. 61/180,160, filed on May 21, 2009, the entirety of which is incorporated herein by reference for any and all purposes.

### TECHNICAL FIELD

The present invention relates to the field of nanoscale devices and the field of Raman spectroscopy.

### BACKGROUND

SERS (Surface-enhanced Raman Spectroscopy) is a promising technique for label-free detection and analysis inside cells that is based on the enhancement of Raman scattering in the vicinity of metal nanostructures. Existing within-cell SERS methods are based on introducing gold or silver nanoparticles through endocytosis. These existing methods, however, suffer from a lack of control over nanoparticle position and from nanoparticle aggregation, both of which compromise the methods' effectiveness. Accordingly, there is a need in the art for devices and methods capable of performing SERS analyses on cells without suffering from the shortcomings of existing methods.

### SUMMARY

In meeting the described challenges, the claimed invention first provides a acicular members having a distal end, at least a portion of the distal end of the acicular glass member being surmounted by a population of metallic nanoparticles, metallic shells, core-shell nanoparticles having a dielectric core and a metallic shell, or any combination thereof, the distal end of said acicular glass member having a diameter of less than about 500 nm.

The claimed invention also provides methods of analysis, comprising inserting, across a boundary of a subject, an acicular glass probe having a distal end, at least a portion of the distal end of the acicular glass probe being surmounted by a population of metallic nanoparticles, and the distal end of said acicular glass probe having a diameter of less than about 500 nm; and irradiating the distal end of the acicular glass probe so as to obtain a first surface-enhanced Raman signal.

### BRIEF DESCRIPTION OF THE DRAWINGS

The summary, as well as the following detailed description, is further understood when read in conjunction with the appended drawings. For the purpose of illustrating the invention, there are shown in the drawings exemplary embodiments of the invention; however, the invention is not limited to the specific methods, compositions, and devices disclosed. In addition, the drawings are not necessarily drawn to scale. In the drawings:

FIG. 1 illustrates scanning electron micrographs (SEM) of the SERS-active nanopipette, a) The nanopipette tip covered with gold nanoparticles; b) magnified view of the nanoparticles coverage of the nanopipette about 10 μm away from the tip; c) bare glass surface of the nanopipette;

FIG. 2 illustrates SERS spectra from a cell nucleus (upper graph) and cytoplasm (lower graph) obtained with the SERS-active nanopipette show distinctly different features, the bottom spectrum was collected from the nanopipette tip before insertion, and an 785 nm excitation laser was used—the graphs are offset for clarity;

FIG. 3 illustrates principal component analysis of the reproducibility of the SERS spectra obtained with the nanopipette from the cytoplasm different cells using 785 nm and 633 nm excitation lasers, each point represents a SERS spectrum—the distance between two data points is proportional to the degree of their correlation, and to provide a reference, the inset graph shows the SERS spectra corresponding to two data points in the principal component space;

FIG. 4 illustrates intracellular monitoring of HeLa cell response to treatment with KCl aqueous solution with SERS-active nanopipette, the time-dependent variation of the cytoplasmic signal is observed, data acquisition time was 20 s, using a 633 nm excitation laser—the spectra are offset for clarity;

FIG. 5 depicts the use of a probe according to the claimed invention, superimposed above a sample Raman spectrum evolved from such use;

FIG. 6 illustrates (A) Scanning electron micrograph (SEM) of the gold colloid used for fabricating the SERS-active nanopipette and its extinction spectrum (B);

FIG. 7 illustrates SEM micrographs of the planar glass substrates coated with gold nanoparticles, the planar SERS substrates were used as a model system for finding the optimal nanoparticle density, corresponding extinction and SERS spectra of the substrates are shown in FIGS. 8 and 9, respectively—the density of the attached nanoparticles is proportional to the time the glass substrates were immersed in the gold colloid, and samples shown in FIG. 7 (a)-(d) correspond to 30 min, 2 hours, 4 hours, and 5 hours of immersion, respectively;

FIG. 8 illustrates UV-VIS extinction spectra of the planar SERS substrates;

FIG. 9 illustrates SERS spectra of poly-l-lysine on gold coated planar substrates with different surface density of the nanoparticles collected with (A) 633 nm excitation laser, (B) 785 nm excitation laser, the (a)-(d) graphs correspond to the samples shown in FIG. 7, (a)-(d), at the lowest nanoparticle density (a), no SERS spectra are detected at both wavelength. Increasing interparticle distance results in appearance of the SERS spectra (b, c)—at the highest particle density the SERS signal obtained with 633 nm excitation laser becomes weaker as concluded from the increased spectral noise (spectrum d, graph A). By contrast, when the 785 nm laser is used on the same sample, the intensity of the SERS signal is significantly better (spectrum d, graph B), which may—without being bound to any particular theory—can be explained by the presence of the clustered gold nanoparticles (Supplementary FIG. 7, d) which stipulates the red shift of the plasmon resonance, responsible for electromagnetic SERS enhancement, and thus the 633 nm laser is not sufficient for exciting the plasmon resonance at the given nanoparticle density and size.

FIG. 10 illustrates SERS fingerprints of intact HeLa cells in suspension (top), isolated HeLa mitochondria (middle), and isolated HeLa nuclei (bottom) obtained on a planar substrate. Each sample has been shown to have the characteristic SERS features;

FIG. 11 illustrates a nanopipette tip interrogating cells in a Petri dish.

FIG. 12 illustrates confocal fluorescent images of the live HeLa cell cytoskeleton actin before (a) and after (b) insertion of the SERS-active nanopipette. The corresponding differential contrast images are shown in panels (c) and (d), respectively. The arrow shows the place of the probe entrance in the cell;

FIG. 13 illustrates calcium response to the nanopipette insertion in the cytoplasm;

FIG. 14 illustrates (a) PCA of the data obtained with SERS-active nanopipette with poly-L-lysine (squares) and HeLa cells (triangles). The data was collected with the 633 nm excitation laser. To provide a reference, the inset graph shows the SERS spectra corresponding to two data points in the principal component space. (b) Pareto chart showing the percentage of information about the original data corresponding to each principal component;

FIG. 15 depicts a schematic of measuring cell response to the change in osmotic pressure with SERS-active nanopipette before (a) and after (c) treating cells with aqueous solution of KCl. Panel (b) shows the SERS spectrum collected from the nanopipette tip inserted in the HeLa cell cytoplasm. The representative time-resolved spectra showing HeLa cell response to treatment with aqueous solution of KCl measured with the SERS-active nanopipette are depicted in panel (d). Time-dependent variation of the cytoplasmic signal has been observed. The dynamic changes in the SERS spectra represent the cell activity in response to the osmotic changes. The spectra in (d) are offset for clarity;

FIG. 16 depicts a comparison of the tip geometry (a-c) and navigation schematics for cell interrogation (d-e) for different SERS probes. SEM of (a) SERS-active nanopipette, (b) fiber optic probe coated with silver nanoparticles, (c) TERS silver probe prepared by electrochemical etching. Panel (d) shows a SERS-active nanopipette interrogating a cell. The nanopipette allows for controlled insertion at different angles, whereas AFM-based TERS probe (e) permits cell penetration only at one angle; and

FIG. 17 illustrates (a) as-produced CNT-tipped cellular probe. (b) SERS-active CNT-tipped pipette functionalized with gold nanoparticles. (c) SERS spectra of HeLa cell homogenate on a SERS-active CNT-tipped pipettes.

### DETAILED DESCRIPTION OF ILLUSTRATIVE EMBODIMENTS

The present invention may be understood more readily by reference to the following detailed description taken in connection with the accompanying figures and examples, which form a part of this disclosure. It is to be understood that this invention is not limited to the specific devices, methods, applications, conditions or parameters described and/or shown herein, and that the terminology used herein is for the purpose of describing particular embodiments by way of example only and is not intended to be limiting of the claimed invention. Also, as used in the specification including the appended claims, the singular forms “a,” “an,” and “the” include the plural, and reference to a particular numerical value includes at least that particular value, unless the context clearly dictates otherwise. The term “plurality”, as used herein, means more than one. When a range of values is expressed, another embodiment includes from the one particular value and/or to the other particular value. Similarly, when values are expressed as approximations, by use of the antecedent “about,” it will be understood that the particular value forms another embodiment. All ranges are inclusive and combinable.

It is to be appreciated that certain features of the invention which are, for clarity, described herein in the context of separate embodiments, may also be provided in combination in a single embodiment. Conversely, various features of the invention that are, for brevity, described in the context of a single embodiment, may also be provided separately or in any subcombination. Further, reference to values stated in ranges include each and every value within that range.

In a first aspect, the present invention provides probes. Probes according to the invention suitably include an acicular member having a distal end, at least a portion of the distal end of the acicular glass member being surmounted by a population of metallic nanoparticles, metallic shells, core-shell nanoparticles having a dielectric core and a metallic shell, or any combination thereof, the distal end of said acicular glass member having a diameter of less than about 500 nm.

The acicular member is suitably made of glass, quartz, carbon, or a combination. In some embodiments, the acicular member is cylindrical; in others, the member has one or more flat sides or faces. The tip of the member may be sharp or blunted, and may even include a dulled or flat face. The member may be tapered or needle-like; but may also be straight and have little to no taper. In some variations, the acicular member is hollow or includes a lumen having a diameter of from about 50 nm to about 800 nm, or from 100 nm to about 500 nm, or even about 350 nm.

The metallic nanoparticles, metallic shells, or core-shell nanoparticles having a dielectric core and a metallic shell of the disclosed probes suitably include Au, Ag, Cu, Pt, Fe, Ph, Pd, Co, Ni, In, Ga, Na, Al, Cd, Hg, Li, O, silica, polystyrene, and the like. Materials suitable for use in SERS analyses are preferable.

In some embodiments, the distal end of the acicular glass member comprises one or more negative charges. Such charges may be achieved by, for example, attaching poly-l-lysine to the member. In some variations, one or more of the metallic nanoparticles, metallic shells, or core-shell nanoparticles having a dielectric core and a metallic shell comprises one or more positive charges. In some embodiments, one or more of the nanoparticles or shells is secured to the distal end of the acicular glass member by electrostatic interaction. Nanoparticles or shells may also be affixed to the acicular glass member by embedding or by fixation with an adhesive or bonding material, depending on the user's needs.

The nanoparticles or shells suitably have a cross-sectional dimension (e.g., diameter) in the range of from about 20 nm to about 500 nm, or even from 50 nm to about 200 nm, or even from about 100 nm to about 150 nm. The nanoparticles or shells are suitably spherical in configuration, though they may also be oblong or of other geometric configuration. Their optimal size and configuration will depend on the needs of the user and various process parameters; the user of ordinary skill will encounter little difficulty in optimizing the particle size to a particular application.

The nanoparticles or shells are suitably present on the probes at a density of from about 1 particles/μm² to about 2,500 particles/μm², or from about 200 particles/μm²to about 1000 particles/μm², or even at about 500 particles/μm². The nanoparticles or shells may be present on the probes at an essentially uniform density, or may be present at a density that varies by location on the probe.

In some embodiments, the probe includes a Raman spectrometer and a source of radiation. Such spectrometers and sources of radiation are commercially available. The probes may also include one or more devices capable of controllably positioning the acicular glass member. Such devices may be motors, manipulators, piezoelectric devices, and the like, and may be manually or automatically controlled. In some embodiments, the positioning device is governed by a controller that has, as an input, a SERS signal from the probe. Such embodiments are useful in controllably positioning the probe in response to real-time (or recorded) SERS signal evolved from the probe.

The claimed invention also provides methods of analysis. These methods suitably include inserting, across a boundary of a subject, an acicular glass probe having a distal end, at least a portion of the distal end of the acicular glass probe being surmounted by a population of metallic nanoparticles, and the distal end of said acicular glass probe having a diameter of less than about 500 nm; and irradiating the distal end of the acicular glass probe so as to obtain a first surface-enhanced Raman signal.

Subjects suitable for the claimed methods include cells, organelles, organs, and the like. A subject boundary may be, for example, a cell wall, a cell membrane, the membrane or wall of an organelle, and the like. Probe insertion may be accomplished manually, or by a motor or other device, as described herein.

In some examples, the methods include introducing to the cell a first agent and irradiating the distal end of the acicular glass probe so as to obtain a second surface-enhanced Raman signal. The introduction of the agent may be accomplished by exerting the first agent across a lumen of the probe, or by introducing the agent to the subject by way of injection, osmosis, sonication, electroporation, or by otherwise exposing the subject to the agent.

The methods may also include comparing the first and second surface-enhanced Raman signals to one another. In this way, the second surface-enhanced Raman signal can be correlated to the presence of the one or more agents, to determine whether the presence of the agent or agents has any effect on the Raman signal of the subject. The user may also add an additional amount of the first agent, adding an amount of a second agent, or both, in response to the second surface-enhanced Raman signal. The additional agent may be added by exerting the first agent, the second agent, or both, across a lumen of the probe, or by adding the agent in one of the other manners described herein. In this way, the methods may be used to deliver a controllable amount of an agent to a subject based on the subject's Raman signal.

For example, if the user knows a priori that the necessary concentration of a therapeutic agent corresponds to a particular Raman signal and that the initial dosage of that agent does not elicit that particular signal, an additional amount or amounts of that agent can be delivered such that that the subject exhibits the desired Raman signal. Similarly, if administration of a particular agent results in a Raman signal known to represent an undesirable state, the user may then, based on that Raman signal, administer a second agent that counteracts the first agent so as to undo any deleterious effects the first agent may have on the subject. A user may use the Raman signals to develop a concentration curve that represents the signal evolved from a subject at various agent dosage levels. In this way, the user may determine the agent concentration present in a subject by comparing the signal evolved from the subject against the calibration curve.

The methods may also suitably include inserting the acicular glass probe across a second boundary of the subject, and irradiating the distal end of the acicular glass probe to obtain a second surface-enhanced Raman signal. The user may then compare the first and second surface-enhanced Raman signals so as to determine the position of the probe relative to one or more of the subject's boundaries. In this way, the user may use the Raman signals evolved at one or more of the probe's positions to determine whether that position corresponds to a position within or outside of a particular boundary of the subject.

### ADDITIONAL DESCRIPTION AND NON-LIMITING EMBODIMENTS

SERS-active probes allow navigation inside cells by monitoring the Surface-Enhanced Raman scattering signal from its tip. The claimed invention demonstrates that the probes can be used for in situ monitoring of cell function in real time. The developed probe is suitable for highly sensitive chemical analysis of nanoliter volumes of materials that are available in only low concentrations.

In one embodiment, SERS-active probes according to the claimed invention are used for in situ intracellular analysis. This probe is based on traditional glass pipettes known in biology. SERS functionality is added by incorporating nanoparticles on the pipette tip. It has been demonstrated that this allows the user to track the location of the tip within the cell. For example, it is shown that the SERS spectra obtained with a probe according to the claimed invention from within the nucleus are different from those obtained within the cytoplasm, and contain typical features associated with DNA. The probe can also monitor cell function in real time.

SERS analysis based on endocytosis of nanoparticles is applied to imaging DNA in single cells along with in situ studies of individual endosomes formed around a gold nanoparticle taken up by a cell. Aggregation of nanoparticles poses difficulties because SERS signals are sensitive to nanoparticle configuration and position. Targeting of nanoparticles to specific location within cells through functionalization of the nanoparticles may interfere with SERS analysis. Further, particles are difficult to remove from the cell, and may have negative effects on cell functionality over an extended term.

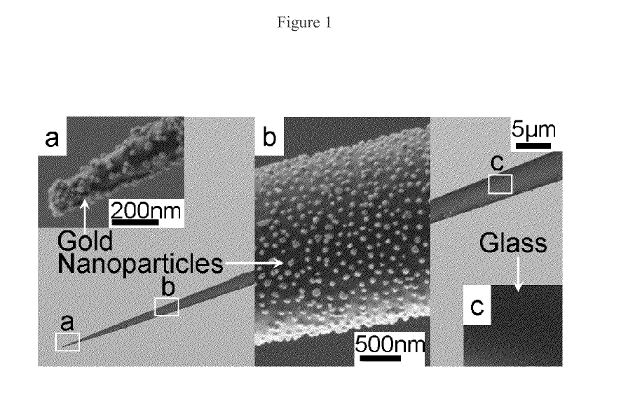

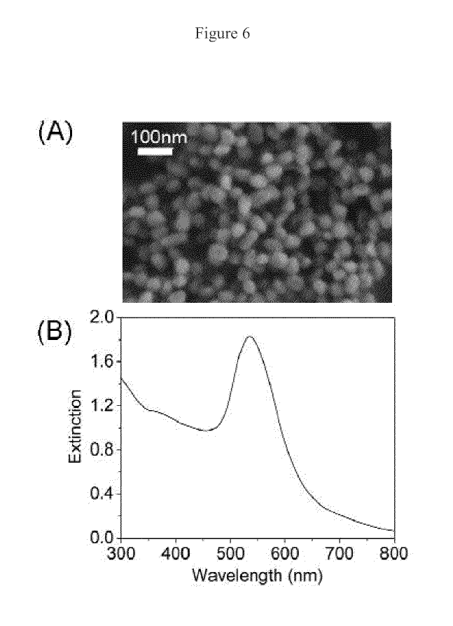

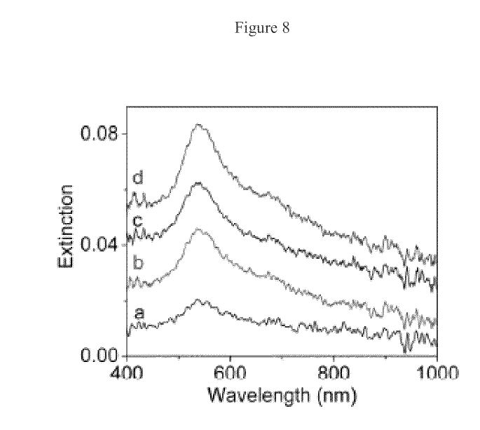

An exemplary SERS-active probe (e.g., a nanopipette) includes a hollow glass capillary with a ca. 150 nm tip coated with gold nanoparticles. FIG. 1 shows a scanning electron micrograph (SEM) of this probe. As shown in FIG. 1 (a) and (b), coverage of the gold nanoparticles is uniform, although the invention includes non-uniform or varying nanoparticle coverage. Surface density of gold nanoparticles determines the characteristics of the SERS-activity of the probe. The relationship between the surface density of gold nanoparticles on a glass substrate and the corresponding SERS enhancement was studied (see FIGS. 6-9).

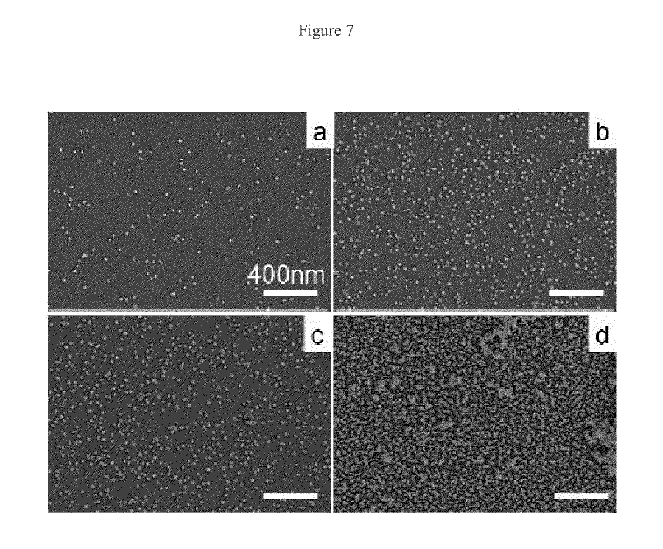

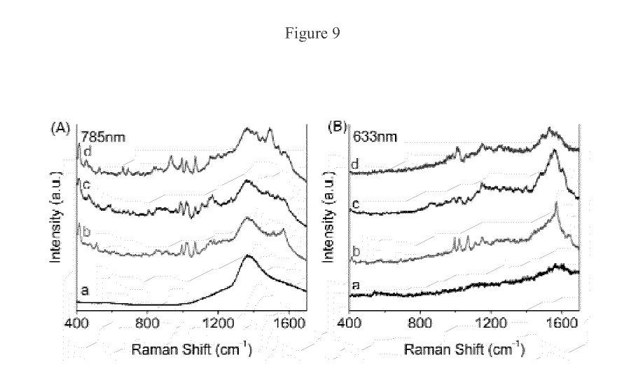

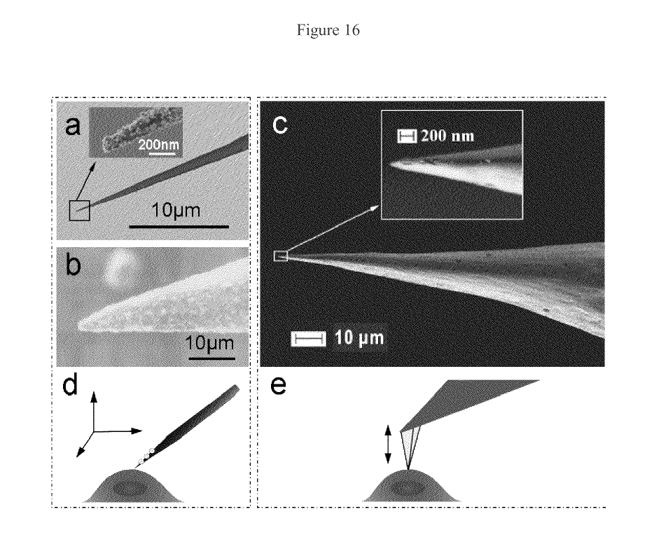

A model system was constructed using planar glass slides coated with gold nanoparticles. SEM images of these substrates with different nanoparticle surface densities are shown in FIG. 7. UV-VIS absorption spectra of all 4 substrates are similar to that of the nanoparticle colloid with the maximum absorption at about 540 nm (see FIG. 16). The corresponding SERS spectra, collected with 633 nm and 785 nm excitation lasers, are demonstrated in FIG. 9. At the lowest nanoparticle density (a), no SERS spectra were detected at either wavelength. Decreasing interparticle distance results in the appearance of the SERS spectra (b, c). At the highest particle density, the SERS signal obtained with 633 nm excitation laser became weaker, as concluded from the increased spectral noise (spectrum d). When a 785 nm laser was used to excite SERS of the same sample, the intensity of the SERS signal was higher (spectrum d).

Without being bound to any particular theory, this may be explained by the presence of the clustered gold nanoparticles that stipulates the red shift of the plasmon resonance, responsible for electromagnetic SERS enhancement. Therefore, the 633 nm laser was less effective for exciting the plasmon resonance at the given nanoparticle density and size. Without being bound to any single theory, these results suggest the average distance between the nanoparticles is suitably smaller than their diameter in order to achieve good SERS enhancement with a 633 nm excitation laser. When the surface density of the nanoparticles becomes very high and the particles form clusters, the plasmon resonance shifts to the longer wavelengths. In this case, SERS enhancement was higher with the 785 nm excitation laser.

It has been previously been shown that 1 μm polygonal gold nanoparticles provide good SERS enhancement. However, for intracellular applications, 1 μm polygonal gold nanoparticles are too large and may cause cell damage.

In non-limiting embodiments of the claimed invention, spherical gold nanoparticles were used, although it is not essential that the particles or shells be perfectly spherical. The average size of the nanoparticles (54 nm) was chosen as a result of the trade-off between the SERS sensitivity, which may use nanoparticles in the 30-100 nm range, and the final size of the probe, which is suitably small enough to minimize effects on the cell during the probe insertion. At the same time, SEM analysis showed that the nanoparticles are strongly attached to the glass surface, which is advantageous in that the particles resist peeling off and remain inside a cell during probe insertion or removal. A scanning electron micrograph of these gold nanoparticles is shown in FIG. 6(A), along with the corresponding UV-VIS absorption spectrum, 6(B). The diameter of the nanoparticles was measured using both SEM images and Zetasizer data.

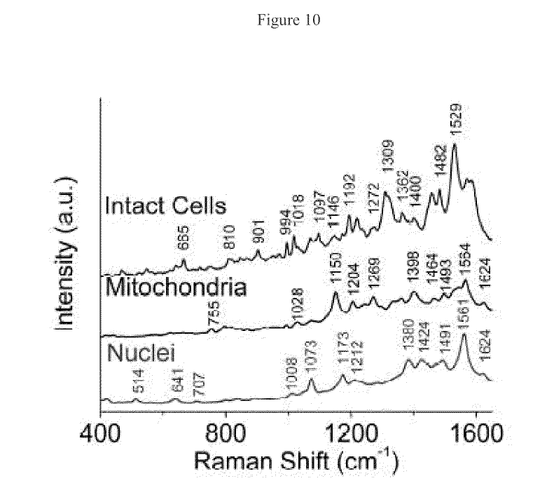

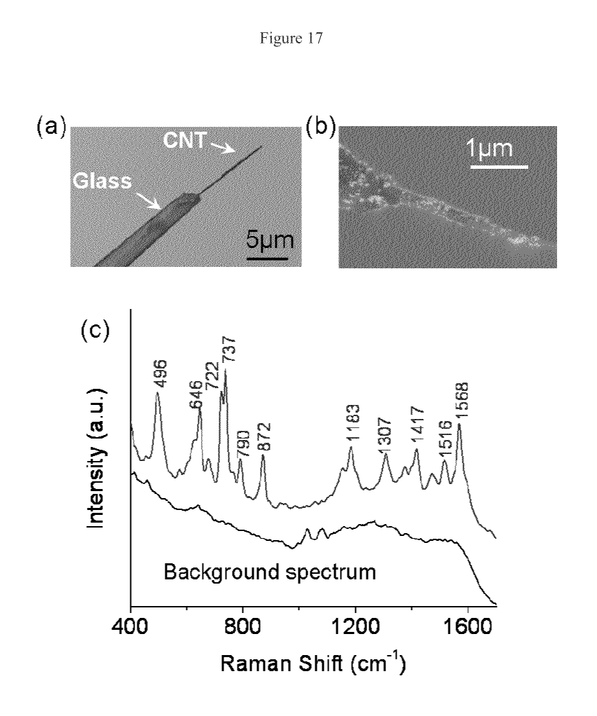

Prior to fabricating the probe, the SERS performance of the model planar substrates was tested on intact HeLa human cervical carcinoma cells. In addition, SERS signatures of isolated HeLa cell nuclei and mitochondria were collected on the model substrates to confirm their specificity for cell studies (see FIG. 10). SERS signatures of isolated HeLa cell nuclei and mitochondria were collected on the model substrates with 40% nanoparticle surface density in order to confirm their specificity for cell studies. (see FIG. 17). The protocols for organelle isolation and purification are described elsewhere herein.

According to the collected data, the SERS fingerprint of the isolated HeLa nuclei is clearly different from that of the cell mitochondria or the cell membrane. The selected nanoparticle configuration (shape and interparticle distance) ensures the desired specificity for intracellular SERS analysis.

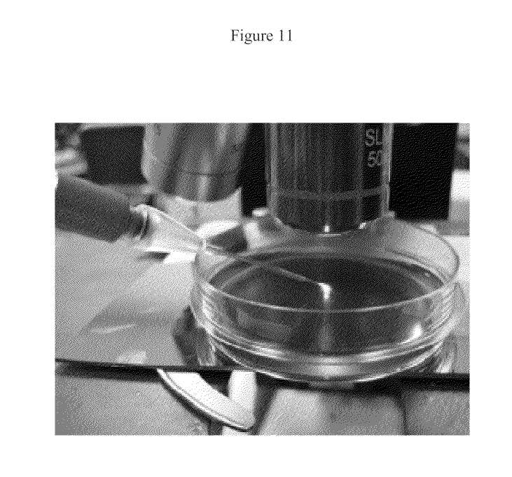

The ability of a probe to provide a SERS signal from a specific location inside a cell was tested on adherent HeLa cells. The experimental setup is shown in FIG. 11. The typical size of these cells was 20 μm.

A SERS-active nanopipette was inserted into a cell following a standard procedure used in cell biology for interrogating adherent cell cultures with glass pipettes. The nanopipette was fixed inside the pipette holder of the Eppendorf InjectMan NI2™ micromanipulator. This micromanipulator allowed precise control over the nanopipette movement. The stepper motor resolution is approximately 40 nm per step, according to the manufacturer.

The nanopipette was positioned above a Petri dish with adherent HeLa cell culture and then directed towards the cells at about a 45° angle. This was continuously monitored under the Raman microscope with a 50× long working distance objective. During the data acquisition, the excitation laser was focused on the nanopipette tip.

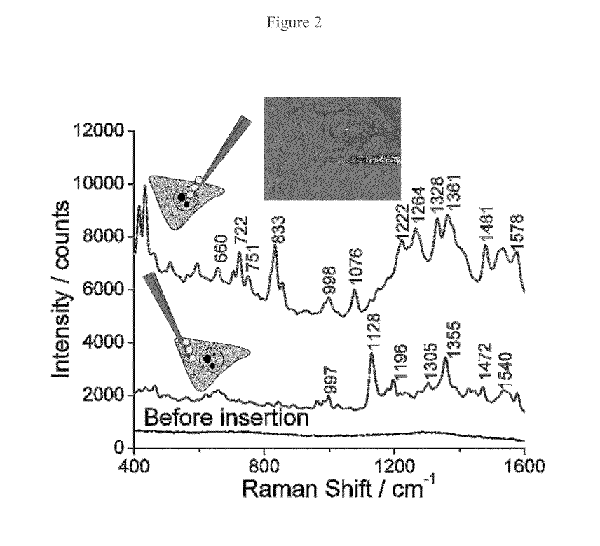

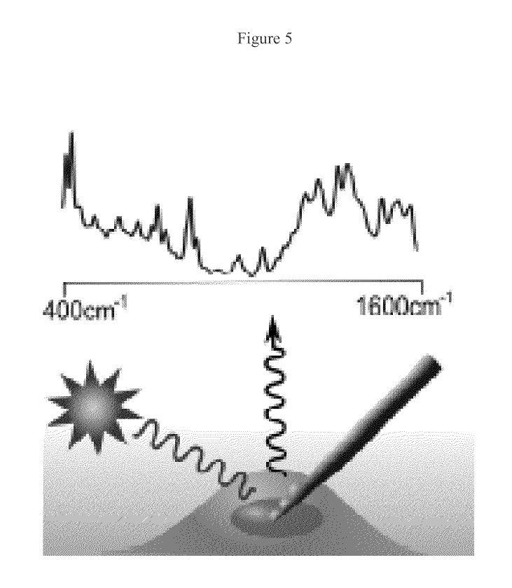

The SERS spectrum collected from the nanopipette tip inserted in the cell nucleus, whose outlines could be observed through regular bright field microscopy, is clearly different from that collected inside the cell cytoplasm (FIG. 2). The data presented in FIG. 5 represents the averages of at least 5 different experiments, conducted on multiple cells with SERS-active nanopipettes. SERS spectra measured from the same location inside a cell demonstrated certain variability in terms of the intensity (10-15%) and, to a minor extent, in the location of spectral peaks. The latter can vary by 10-20 cm−1, which was within the expected range normally observed in SERS.

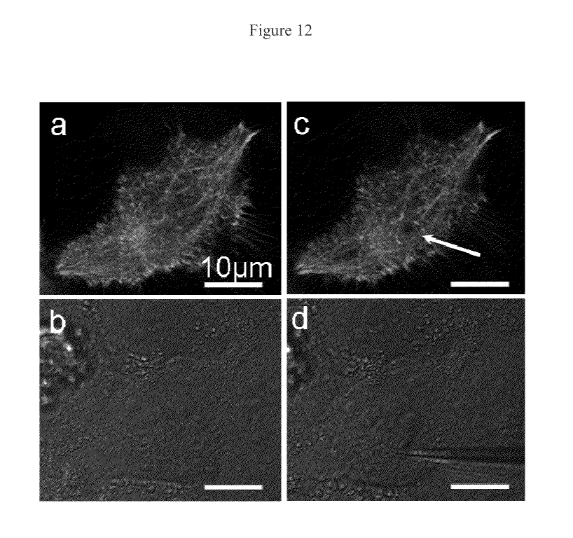

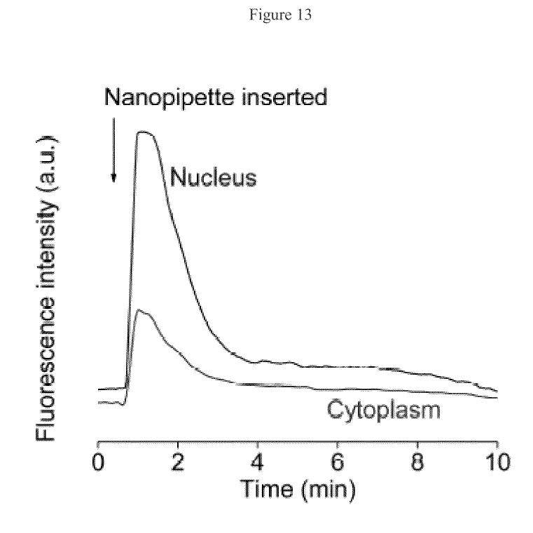

Cell response to probe insertion was thoroughly studied before conducting SERS analysis. To ensure that SERS data did not originate from probe insertion-induced cell signaling, a 20 min recovery period after the insertion was allowed before starting the measurements. (see FIGS. 12 and 13). SERS spectra collected from probe tips inserted in a cell nucleus, whose outlines could be observed through regular bright field microscopy, were different from those collected inside the cell cytoplasm (FIG. 2).

The nuclear spectrum has features that are, without being bound to any particular theory, likely attributable to its high protein and amino acid content (1076 cm⁻¹, 1222 cm⁻¹, 1264 cm⁻¹, 1328 cm⁻¹, 1361 cm⁻¹), and to DNA (660 cm⁻¹, 722cm⁻¹). The cytoplasmic SERS spectrum does not show the DNA bands. However, that spectrum still contains the peaks related to the protein constituents of the cytoplasm (1128 cm⁻¹, 1540 cm⁻¹, 1355 cm⁻¹). At the same time, the 1004 cm⁻¹ and 1198 cm⁻¹ peaks are, without being bound to any particular theory, likely associated with phenylalanine. As shown, the cytoplasmic phenylalanine signal was stronger than that of the nucleus. Probe insertion did not cause fatal damage to cells.

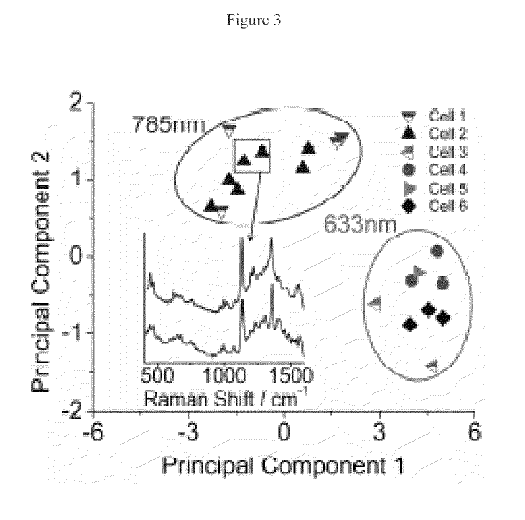

Irregularities of the SERS substrate are, in some instances, blamed for poor reproducibility of SERS spectra. To demonstrate that gold-nanoparticle coverage of the probe surface used in the described analyses provided sufficient regularity to obtain reproducible SERS spectra, Principal Component Analysis (PCA) was used to assay the data reproducibility spectra that were obtained from in situ pipette measurement (FIG. 3).

PCA is a multivariate data analysis widely used in spectroscopy for facilitating data interpretation by reducing its dimensionality and calculating the degree of correlation (similarity) between the spectra (details are described elsewhere herein). The PCA results presented in FIG. 3 demonstrate the SERS spectra collected from multiple different cells with the SERS-active probes were well correlated if the same excitation laser was used.

Intracellular Sensing with SERS-Active Nanopipette

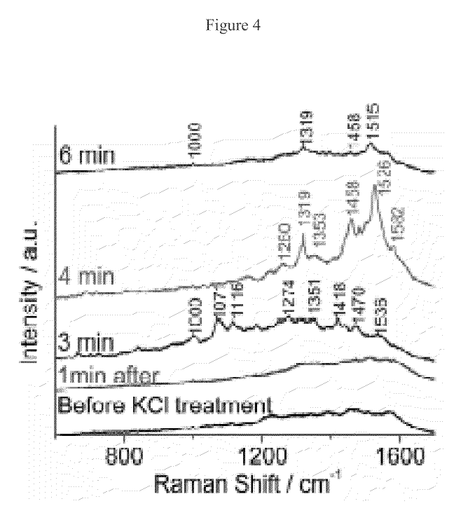

Monitoring cell activity by SERS-active probes upon the application of the external stimulus was also examined Probes were inserted into the cytoplasmic region of a living adherent cell, and the background spectrum was collected (FIG. 4). After 10 minutes, an aqueous KCl solution was added to the cell medium to achieve a final concentration of 55 mM. Time sequence of the SERS spectra from the cell interior was collected (FIG. 4).

Increased levels of extracellular potassium ions cause depolarization of the cell membrane potential due to the decrease in the equilibrium potential for this ion. The loss of cytosolic water, resulting from an increase in environmental osmolarity and plasma membrane depolarization, can lead to alterations in cytosolic concentration of cellular colloids such as proteins and organic phosphates, and the hydration level of proteins. The hydration state of cellular components and resulting conformational modifications of proteins are the likely cause of the observed SERS signal modulation. This was indicated by the appearance of high intensity peaks in the 1200-1500cm⁻¹ region (FIG. 4).

SERS has been applied for monitoring the uptake of dilute solution of doxorubicin (an antitumor drug) by a living cancer cell. Actual physiological cellular response to a pharmaceutical compound or any external stimulus application has not been assessed using in situ SERS. Herein is described an assessment of real time cell response to treatment with an aqueous solution of KCl.

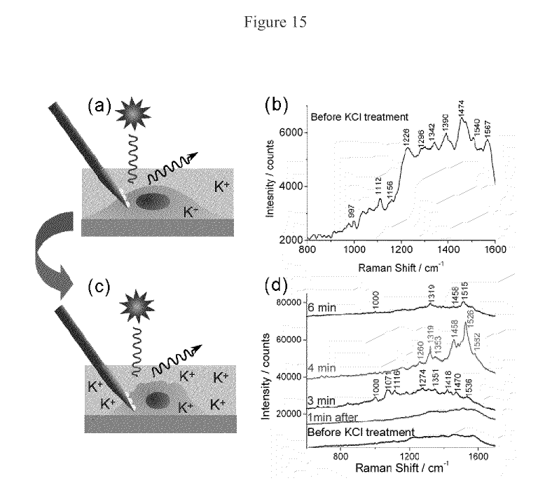

The nanopipette was inserted into the cytoplasmic region of a living adherent cell, and the background spectrum was collected (FIG. 15b). After that, an aqueous KCl solution was added to the cell medium to achieve a final concentration of 55 mM. A time sequence of the SERS spectra from the cell interior was collected (FIG. 15d). KCl was used to trigger cell activity by providing the external cell stimulus. The configuration of the gold nanoparticles was not affected by KCl, as the nanoparticles were fixed on the nanopipette surface.

Treated cells exhibited almost a 5-fold increase in the Raman scattering intensity, compared to the Raman spectra obtained on untreated cells, as can be seen from comparing FIGS. 15b and 15d. The maximum intensity amplitude of SERS spectra before and after KCl treatment was about 4000 and 20000 CCD counts, respectively (FIG. 15d). Due to this fact, the SERS spectrum collected from the nanopipette tip inserted in the HeLa cell cytoplasmic region appears almost featureless compared to the spectra collected after the treatment with KCl solution (FIG. 15d), when plotted without intensity normalization.

From a biological point of view, increased levels of extracellular potassium ions likely cause depolarization of the cell membrane potential due to the decrease in the equilibrium potential for this ion. The loss of cytosolic water, resulting from an increase in environmental osmolarity and plasma membrane depolarization, can lead to alterations in the cytosolic concentration of cellular colloids, such as proteins and organic phosphates, and the hydration level of proteins.

The hydration state of cellular components and the resulting conformational modifications of proteins may be one cause of the observed SERS signal modulation. This is suggested by the appearance of high intensity peaks in the 1200-1500 cm−1 region (FIG. 15b). The intensities of certain spectral peaks exhibited a dynamic behavior after the KCl treatment.

One possible reason for the appearance of 1319 cm−1, 1260 cm−1, 1515 cm−1, and 1526 cm−1 SERS peaks at different time points can be associated with the induced expression of various types of stress proteins. The change in environmental osmolarity triggers the cellular adaptive mechanism, which leads not only to the induction but also to the suppression of specific proteins. This process occurs primarily in the first several minutes after the addition of KCl to the cell medium. The dynamics of this complex mechanism manifests itself in the recorded SERS spectra. Alterations of the peak profile at different time points reflect dynamic cellular processes in response to perturbations of extracellular environment. After 6 minutes, the cell volume regulatory mechanism restores the initial iso-osmotic state of the cell. This is reflected in the SERS spectrum, which becomes again similar to that collected before the cell treatment with KCl.

These results demonstrate that the SERS-active nanopipette works as a real time sensor of local intracellular biochemical processes. It is critical to emphasize that this experiment was performed without adding any labels to the cell and the cell activity was monitored in situ. The level of chemical sensitivity offered by SERS is superior to that of any other currently available biological techniques. These results can be further extended to combining the basic nanopipette fluid delivery function with the SERS sensing. By controlling the injection pressure and time, one can deliver femtoliters of fluid into a cell and simultaneously assess the cell response in real time. Using electrostatic driving force to motivate specific molecules to the SERS substrate could further improve the performance of the SERS-active nanopipette. Combination of this method with the nanopipette allows one to selectively target different molecules in living cells with a higher level of selectivity. The applications of a SERS-active nanopipette are not limited to cellular studies, and the nanopipette also enables highly localized chemical analysis of low concentration chemicals, which is useful in micro-analytical chemistry, environmental and forensic studies.

Experimental

A SERS-active probe for in situ intracellular observations was constructed and studied. It was demonstrated that positioning of the probe tip either within the cell nucleus or cytoplasm could be clearly distinguished through the measured SERS. Feedback on positioning of the probe tip within cells provides valuable information during cell injections, for single cell surgery or for in situ study of cell signaling. Robust reproducibility of cell SERS signal was obtained suggesting the possibility of distinguishing the proximity of the tip to other cell organelles and concentration of various molecular species. For the first time, in situ cell response to the changes in its environment was measured by using an intracellular SERS probe. Applications of a SERS-active probe are not limited to cellular studies, and the probe also enables highly localized chemical analysis of low concentration materials, which is useful for micro-analytical chemistry, environmental and forensic studies.

Synthesis of gold nanoparticles. Gold colloid was synthesized using the Turkevitch method. The protocol was modified to optimize the size of gold nanoparticles. Hydrogen tetrachloroaurate (HAuCl₄) aqueous solution (10 ml, 2.5 mM concentration) was boiled and then 2 mL of sodium citrate was added with vigorous agitation. The mixture was stirred until becoming deep red in color, then removed from the heat. After cooling down, the colloid was left to reach equilibrium in the dark for 1 week. This protocol yielded gold nanoparticles with the average diameter of 54 nm as confirmed by scanning electron microscopy analysis. Zeta potential, related to the surface charge of the nanoparticles, was measured to be approximately −40 mV.

Fabrication of the SERS-active probes. Glass probes were prepared by pulling a hollow borosilicate glass capillary to a 150 nm tip diameter. The characteristics of the glass capillary are as follows: length 10 mm, inner diameter 0.75 mm, outer diameter 1 mm. The glass capillaries were purchased from Sutter Instrument (BF100-78-10). The dimensions of the resulting probe were determined by the parameters on the micropipette puller (Laser based micropipette puller P-2000, Sutter Instrument, USA). After pulling, the glass pipettes were soaked in a mixture of 95% ethanol and 1M aqueous solution of sodium hydroxide for 1 hour. After washing with 15 MΩ deionized water, the pipettes were left to dry at room temperature. The pipettes were then dip-coated with 0.001 wt % aqueous solution of poly-l-lysine. Polymer coating enabled immobilization of gold nanoparticles on the glass due to the electrostatic interaction between the positively charged terminal NH₂ groups of the poly-l-lysine and negatively charged Au nanoparticles.

Characterization techniques. Raman spectroscopy analysis was performed using a micro-Raman spectrometer (Renishaw, RM 1000/2000) in conjunction with an Ar⁺ 514.5 nm gas laser, 632.8 nm HeNe laser (1800 lines/mm grating), and a diode laser operating at 785 nm wavelength (1200 lines/mm grating). The laser source was focused on the sample through a long working distance 50× objective to a spot size of approximately 2 μm. The acquisition time for all spectra was 10-20 s. Data analysis was performed using the Wire 2.0 software. SEM images were collected with the field emission scanning electron microscope Zeiss Supra 50VP. Before imaging, the gold-functionalized glass slides were sputter-coated with 2 nm of Pt/Pd. The images were collected at 5 kV accelerating voltage. SEM images of the SERS-active probe were acquired at 0.7-2 kV accelerating voltage without any conductive coating. UV-VIS absorption spectra were acquired using a UV-VIS spectrophotometer (Thermo Scientific, Evolution 600). The zeta potential of gold nanoparticles was measured using a Zetaziser Nano ZS (Malvern Instruments, UK). Confocal fluorescence microscopy was carried out using the Olympus FluoView 1000 microscope.

Data analysis. Principal component analysis (PCA) is a method of analyzing complex sets of data with multiple variables. The technique facilitates identification of hidden relationships between data sets by reducing their dimensionality and representing the data in the new coordinate system. Raman spectrum can be considered as a data matrix where the first column represents the Raman shift and the second column contains the corresponding signal intensity. For PCA of n spectra with p data points, an n-by-p matrix is constructed where each row represents a Raman intensity spectrum. The purpose of the PCA is to find a new p-dimensional orthogonal coordinate system where the data projection on each coordinate axis has a sequentially maximal variance. Each projection corresponds to a linear combination of the original data with the first projection having the maximum variance and representing the first principal component.

A proof-of-concept experiment was conducted with the planar substrates to check their capability of providing distinct SERS fingerprints of cells and cell organelles. This crucial characterization of the substrate is required for creating a SERS-active probe for intacellular analysis. SERS spectra of intact HeLa cells, isolated nuclei, and mitochondria are shown in FIG. 10. The results clearly show that the characteristic SERS spectra of the cell organelles can be measured with the given SERS-active system.

Studying cell activity with the SERS-active probes requires understanding of the cell's response to the probe's insertion to avoid the uncertainty in the origin of the SERS signal. It has been previously demonstrated that mechanical stress induced by applying force to a cell, causes the elevation of the intracellular calcium Ca²⁺ by activating mechanosensitive ion channels. The triggering mechanism of this phenomenon have been explained by the deformation of the cytoskeleton upon the application of force to a cell membrane. Therefore, the effect of the SERS-active probe insertion on the cytoskeleton network configuration was analyzed in a living HeLa cell. An EYFP-fused β-actin expression construction was transfected into HeLa cells, and the produced fusion fluorescent protein was incorporated into the cytoskeleton. The confocal fluorescent image of the intact HeLa cell cytoskeleton is shown in FIG. 12, (a).

Insertion of the SERS-active probe causes only a localized deformation of the actin filaments without damaging the rest of the cytoskeleton network (FIG. 12, (b)). Thus, without being bound to any particular theory, the probe insertion should not evoke significant cell signaling activity. However, since the localized deformation of the cytoskeleton upon the probe insertion still occurs, it is, without being bound to any particular theory, expected that a Ca²⁺ signaling mechanism will be triggered, leading to a change in the intracellular Ca²30 concentration Moreover, SEM analysis showed that the nanoparticles are strongly attached to the glass surface, so none of the particles peel off and remain inside a cell during the nanopipette insertion and after its removal from a cell. Microscopic analysis showed that the cells remain viable after the nanopipette withdrawal.

To confirm this, living HeLa cells were treated with Fluo4 fluorescent dye which binds to the free cytosolic and nuclear calcium. The intensity of the fluorescence probe, which is proportional to the intracellular calcium concentration, was monitored by a confocal laser scanning microscope inside the cell cytoplasm and the nucleus, separately. FIG. 13 shows the cell Ca²⁺ response when the probe is inserted in the cytoplasmic region. The probe insertion triggers different response in the nucleus and the cytoplasm due to the existence of the separate calcium signaling networks in both parts of a cell. Nuclear Ca²⁺ concentration increase is larger and takes longer to return to the basal level when the cell metabolic activity adjusts to the presence of the probe (appx. 10 min). In the case when the probe was inserted inside the nucleus, the recovery time was on the order of 15-20 min. For SERS measurements, a 20 min recovery period was allowed before the data was collected. This ensured that the SERS data did not originate from probe insertion-induced cell signaling.

Data Reproducibility

Irregularities of the SERS substrate are considered one reason for the poor reproducibility of SERS spectra apart from spectral blinking. The irreproducibility problem can be solved by creating SERS substrates with highly uniform metal nanostructures.

One example is the nanosphere lithography technique which has been successfully applied for creating SERS active substrates and obtaining highly reproducible spectra of various materials. However, in the case of biological and especially cellular SERS studies, special consideration is given to the problem of spectral reproducibility.

It is advantageous to have a SERS substrate with a uniform configuration of metal nanostructures. It is also useful to understand that a highly sensitive SERS sensor that allows detection of compositional changes in the intracellular environment with a submicrometer resolution will provide different SERS spectra from different locations inside a cell due to the cell heterogeneity. The differences between the spectra should still be within the same range if the same cell compartment is being analyzed.

To test the performance of the SERS-active nanopipette, data obtained with the SERS-active nanopipette from a pure chemical (poly-1Llysine) and the heterogeneous HeLa cell cytoplasm were compared. The spectra were collected from multiple cells.

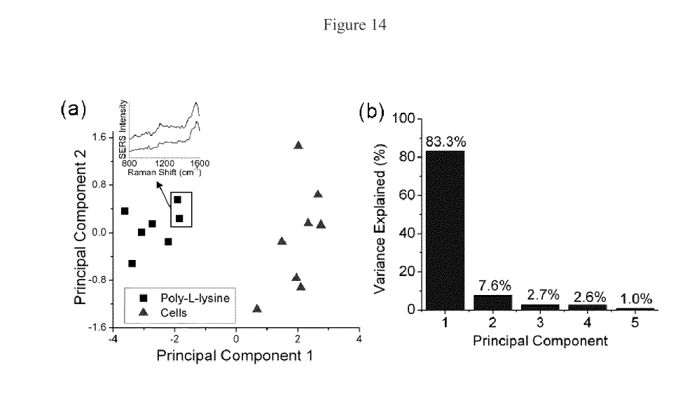

The results are presented in the principal components space in FIG. 14(a). Principal component analysis is a multivariate data analysis technique that is widely used in spectroscopy for facilitating data interpretation by reducing its dimensionality and calculating the degree of correlation (similarity) between the spectra.

The data shown in FIG. 14 show the original SERS spectra mapped into the new coordinate system defined by the principal components. Here the principal components represent a coordinate system rather than a projection of the original data on the new axes. The results of the data analysis demonstrate that the data scatter for a pure chemical is lower (small variation of both components) than that for the cell cytoplasm (small variation of component 1, but a larger variation of component 2). At the same time, the spectra from the HeLa cell cytoplasm are located within the range.

A Pareto chart shown in FIG. 14(b), demonstrates that the first principal component provides about 83% of information about the variability of the original data. The second principal component corresponds to only 7.6% of the original data variability. As a result, the variation of the second principal component observed is FIG. 14(a) is less significant than that of the first principal component.

Accordingly, the SERS nanopipette provides reproducible data. Repeatability of the data obtained with different nanopipettes is within the range observed for different cells, and the slight variation in the interparticle distance for different probes does not cause a significant signal variation.

SERS-Active Nanopipette

After establishing a suitable interparticle distance, the SERS-active nanopipette was fabricated. The nanopipette is comprised of a hollow glass capillary with a ˜100-500 nm tip and is coated with gold nanoparticles. The overall length of the capillary is on the order of 10 cm and the outer diameter is 1 mm. Glass pipettes with such dimensions can be fitted into a standard micromanipulator and fluid injector, which are used for cell microinjection. SERS-active nanopipettes thus do not not require any specialized equipment and are easily adopted by those in the field.

FIG. 1 shows the scanning electron micrograph (SEM) of the SERS-active nanopipette. As shown in FIG. 1 (a) and (b), the coverage of the gold nanoparticles is uniform. The nanoparticles are fixed on the nanopipette tip, and interparticle distance can be controlled by the nanopipette assembly conditions. The surface density of gold nanoparticles determines the characteristics of the SERS-activity of the nanopipette, as shown by the results presented herein.

Nanopipette fabrication is discussed in detail in the Methods section. Briefly, the glass pipettes are prepared from commercial microcapillaries by laser pulling, then coated with a poly-l-lysine polymer layer that contains positive NH₂ functional groups. At the last step, the nanopipettes are coated with negatively charged gold nanoparticles, which bind to the polymer from the colloid through the electrostatic interaction. The interaction time between the nanoparticles and the pipette surface along with the nanoparticles colloid concentration are the major parameters controlling the nanoparticle surface density. This functionalization technique can be applied to other substrates, such as, optical fibers. Carbon nanopipettes described can also be transformed into SERS probes.

Supplementary Methods

Cell culture. Monolayer cultures of HeLa cells, a human cervical carcinoma cell line, were grown to 85% confluence in Dulbecco's modified Eagle's medium, supplemented with 10% donor horse serum, 100 U/ml Penicillin, 100 μg/ml Streptomicin, and 1 mM L-Glutamine. Cells were maintained at 37° C. in a humidified, 5% carbon dioxide atmosphere.

Imaging Ca²⁺ in living cells. Changes in the intracellular Ca²⁺ concentration were examined with Fluo-4AM probe. Cells seeded on glass-bottom dishes the day before the experiment were washed with HEPES butler (20 mM HEPES, pH 7.4, 137 mM NaCl, 5 mM KCl, 1 mM KH₂PO₄, 1 mM MgCl₂, 2 mM CaCl₂, 10 mM Glucose) and loaded with 2 Fluo-4AM in the same buffer with 2.5 mM probenecide for 20 min at room temperature. After incubation, cells were washed at least twice and kept in working buffer with probenecide for additional 15 minutes for stabilization. Cell examination showed uniformed distribution of Fluo-4AM throughout the cells, suggesting no compartmentalization of Fluo-4 in the organelles other than nucleus. To detect changes in [Ca²⁺]i, the average fluorescence intensity was measured over the each tested cell in sequential image acquisition mode. 12-bit images were acquired every 5-10 sec with the Olympus Fluoview 1000 confocal laser scanning microscope.

Cytoskeleton labeling. HeLa cells were transfected with cDNA encoding for EYFP-β-actin using GenDrill™ DNA In Vitro Transfection Reagent (BamaGen BioScience LLC, Gaithersburg) according to the manufacturer's instructions. Experiments were performed 24 hours after transfection. Live images of cells expressing enhanced yellow fluorescence EYFP protein were collected with Olympus IX-81 confocal microscope using 525-605 nm band pass emission filter, 488 nm laser was used for excitation.

Mitochondria isolation and purification by continuous Percoll density gradient centrifugation. Mitochondria from about 7×10⁷ HeLa cells were isolated with the Pierce mitochondria isolation kit according to the manufacturer's protocol. Briefly, cells were harvested by trynsinization, washed with PBS, incubated in swelling buffer supplemented with protease inhibitors cocktail, followed by homogenization with a Dounce homogenizer. Unbroken cells, nuclei, and cell debris were removed by two centrifugations at 1000 g for 10 min at 4° C. Mitochondria sample was obtained from the supernatant by centrifugation at 6000 g for 15 min at 4° C.

Mitochondria prepared by differential centrifugation were further purified in Percoll gradient. Pellet was suspended in Mannitol buffer, layered on 30% Percoll gradient and centrifuged at 95000 g max for 30 min in 70.1Ti Backman rotor at 4° C. Fractions of 0.25 ml number 1 to 9 from the bottom of the centrifuge tube were collected and subjected to Western blotting. The mitochondria-enriched fractions, which were free from other cell contaminants were suspended in mitochondria storage buffer (250 mM Sucrose, 10 mM Tris-base, pH 7.4) and used for SERS analysis. Integrity of the mitochondrial outer membrane as an indicator of metabolic functionality of the purified organelles was detected with the Cytochrome c Oxidase Assay Kit (Sigma). 75 cytochrome c oxidase activity was observed demonstrating isolation of a high level of functionally intact mitochondria.

Preparation of isolated nuclei from adherent HeLa cells. The procedure for cell nuclei isolation was based on cells lysis in hypotonic buffer with a low concentration of the non-ionic detergent NP-40. All subsequent manipulations were performed on ice. Cells from one 100 mm plate were grown up to 85% confluence, rinsed with cold PBS, and one time with cold nuclei buffer (NB) containing 10 mM HEPES, pH 7.4, 10 mM KCl, 2 mM MgCl₂. Just before use, 1 mM DTT, 0.75 m M Spermidine, 0.15 mM Spermine, and Protease inhibitors cocktail were added to this buffer. 2 ml of cold NB with 0.2% NP-40 was added to the cultured cells. Cells were scraped and held on ice for 30 minutes to facilitate cell-swelling. Cell suspensions were then homogenized by 60 strokes in a Dounce homogenizer with a loose pestle. Cell lysate was transferred into microfuge tubes and centrifuged twice at 1000 g, 4° C. for 5 min to wash out detergent solution. The pelleted nuclei were resuspended in 100 μl of Nuclei storage buffer containing 10 mM HEPES, pH 7.4, 80 mM KCl, 20 mM NaCl, 1 mM MgCl₂, 250 mM Sucrose. Just before use, 1 mM DTT, 0.75 mM Spermidine, 0.15 mM Spermine, and Protease inhibitors cocktail were added. Phase microscopy analysis showed the nuclei to be free of any observable cellular membrane fragments

A multifunctional probe that allows simultaneous cell injection and intacellular surface-enhanced Raman spectroscopy (SERS) analysis was developed. SERS spectra contain the characteristic frequencies of molecular bond vibrations. This is a unique method for studying cell biochemistry and physiology on a single organelle level. Unlike the fluorescence spectroscopy, it does not require any specific staining. The principle of SERS is based on very large electromagnetic field enhancement localized around a nano-rough metallic surface. Gold colloids are widely used SERS substrates. Previously, the colloidal nanoparticles were introduced into a cell by the mechanism of endocytosis. The disadvantage of this method is the uncontrollable aggregation and distribution of gold nanoparticles inside a cell which causes a significant uncertainty in the origin of the acquired data. At the same time, the nanoparticle uptake is irreversible. The claimed probes, however, enable selective signal acquisition from any point-of-interest inside a cell. The probes are thus capable of providing a localized SERS signal with sub-nanometer resolution in real time.

Comparison of SERS-Active Nanopipette with Other SERS Probes

FIG. 16 (a-c) compares the tip geometry of a SERS nanopipette, 40 fiber optic-based SERS probe, and a typical TERS (Tip-Enhanced Raman Spectroscopy) probe. The SERS nanopipette shown in FIG. 16 (a) has almost cylindrical tip which is optimal for cell probing. The prior art fiber optic probe shown in FIG. 16b has not only a very large tip but is also highly conical. With these dimensions, this probe is not optimal for cell probing and further shape optimization is required. The TERS probe shown in FIG. 16 (c) has very fine tip which ensures high spatial resolution. However, compared to SERS nanopipette, this probe has a large apex angle which would be an obstacle in using it for cell probing. Furthermore, the nanopipette compatibility with standard micromanipulators allows for more freedom in navigating the probe into a cell, in comparison to AFM-based TERS probes. FIG. 16 (d-e) illustrates this fundamental difference between SERS nanopipettes and TERS probes.

Carbon Nanotube-Tipped SERS-Active Probes

A glass pipette with a carbon nanotube attached to its tip was previously developed. The assembly of such probes relies on the use of magnetic field for pulling a magnetic carbon nanotube out of a glass pipette and the fixation of the nanotube on the pipette tip.

FIG. 17 (a) shows an SEM micrograph of as-produced probe. In this work, probes were functionalized with gold nanoparticles in order to enable SERS functionality. The same colloidal gold nanoparticles with uniform size and shape which were used for designing glass-based SERS-active nanopipettes were employed in this work. The resulting CNT-tipped probe coated with gold nanoparticles is shown in FIG. 17 (b). SERS spectrum of HeLa cell homogenate measured using this probe is shown in FIG. 17 (c). It is clearly demonstrated that CNT-tipped nanoprobes functionalized with gold nanoparticles enable sensing of cell biochemical components.

What is claimed:

1. A probe, comprising:
an acicular member having a distal end,
at least a portion of the distal end of the acicular glass member being surmounted by a population of metallic nanoparticles, metallic shells, core-shell nanoparticles having a dielectric core and a metallic shell, or any combination thereof,
the distal end of said acicular glass member having a diameter of less than about 500 nm.

2. The probe of claim 1, wherein the acicular member comprises glass, quartz, carbon, or any combination thereof.

3. The probe of claim 1, wherein the acicular member comprises a lumen having a diameter of from about 50 nm to about 800 nm.

4. The probe of claim 1, wherein one or more of the metallic nanoparticles, metallic shells, or core-shell nanoparticles having a dielectric core and a metallic shell, comprises Au, Ag, Cu, Pt, Fe, Ph, Pd, Co, Ni, In, Ga, Na, Al, Cd, Hg, Li, O, silica, polystyrene, or any combination thereof.

5. The probe of claim 1, wherein the distal end of the acicular glass member comprises one or more negative charges.

6. The probe of claim 5, wherein one or more of the metallic nanoparticles, metallic shells, core-shell nanoparticles having a dielectric core and a metallic shell comprises one or more positive charges.

7. The probe of claim 1, wherein one or more of the metallic nanoparticles, metallic shells, core-shell nanoparticles having a dielectric core and a metallic shell is secured to the distal end of the acicular glass member by electrostatic interaction.

8. The probe of claim 1, wherein one or more of the metallic nanoparticles, metallic shells, or core-shell nanoparticles having a dielectric core and a metallic shell, has a cross-sectional dimension in the range of from about 20 nm to about 500 nm.

9. The probe of claim 1, wherein one or more of the metallic nanoparticles, metallic shells, or core-shell nanoparticles having a dielectric core and a metallic shell, has a cross-sectional dimension in the range of from about 50 nm to about 200 nm.

10. The probe of claim 1, wherein the density of the metallic nanoparticles, metallic shells, or core-shell nanoparticles having a dielectric core and a metallic shell, surmounting the distal end of the acicular glass member is from about 1 particles/μm² to about 2,500 particles/μm².

11. The probe of claim 1, further comprising a Raman spectrometer and a source of radiation.

12. The probe of claim 1, wherein the distal end of the acicular glass member comprises a flat tip.

13. The probe of claim 1, further comprising a device capable of controllably positioning the acicular glass member.

14. A method of analysis, comprising:
inserting, across a boundary of a subject, an acicular glass probe having a distal end, at least a portion of the distal end of the acicular glass probe being surmounted by a population of metallic nanoparticles, and the distal end of said acicular glass probe having a diameter of less than about 500 nm; and
irradiating the distal end of the acicular glass probe so as to obtain a first surface-enhanced Raman signal.

15. The method of claim 14, wherein the subject comprises a cell.

16. The method of claim 15, wherein the boundary comprises a cell wall, a cell membrane, the boundary of an organelle, or any combination thereof.

17. The method of claim 15, further comprising introducing to the cell a first agent and irradiating the distal end of the acicular glass probe so as to obtain a second surface-enhanced Raman signal.

18. The method of claim 17, wherein the introducing comprises exerting the first agent across a lumen of the probe.

19. The method of claim 17, further comprising comparing the first and second surface-enhanced Raman signals.

20. The method of claim 19, further comprising correlating the second surface-enhanced Raman signal to the presence o the one or more agents.

21. The method of claim 20, further comprising adding an additional amount of the first agent, adding an amount of a second agent, or both, in response to the second surface-enhanced Raman signal.

22. The method of claim 21, wherein adding the additional amount of the first agent, adding an amount of a second agent, or both, is accomplished by exerting the first agent, the second agent, or both, across a lumen of the probe.

23. The method of claim 14, further comprising inserting the acicular glass probe across a second boundary of the subject, and irradiating the distal end of the acicular glass probe to obtain a second surface-enhanced Raman signal.

24. The method of claim 23, further comprising comparing the first and second surface-enhanced Raman signals so as to determine the position of the probe relative to one or more of the subject's boundaries.
